(19) United States
(12) Patent Application Publication
(10) Pub. No.: US 2020/0060760 A1
(43) Pub. Date: Feb. 27, 2020

(54) CATHETER SYSTEM WITH A HIGH RADIO FREQUENCY ELECTROMAGNETIC FIELD

(71) Applicant: SPARTAN MICRO, INC., Round Rock, TX (US)

(72) Inventors: Eric P. Stoppenhagen, Round Rock, TX (US); Mark Dias, Round Rock, TX (US)

(21) Appl. No.: 16/529,257

(22) Filed: Aug. 1, 2019

Related U.S. Application Data
(60) Provisional application No. 62/713,099, filed on Aug. 1, 2018.
(60) Provisional application No. 62/713,097, filed on Aug. 1, 2018.

(51) Int. Cl. A61B 18/14; A61B 17/12

(52) U.S. Cl. CPC A61B 18/1492; A61B 17/1214; A61B 2018/00577; A61B 2018/1407; A61B 2017/1209

(57) ABSTRACT

A catheter system that includes a catheter with a lumen and extends from a proximal end to a distal end of the catheter, the distal end of the catheter comprising an opening. The catheter system further includes an RF generator configured to produce an high radio frequencies. An electrode is disposed within a wall of the catheter at the distal end of the catheter. The electrode is configured to produce a high radio frequency electromagnetic field that is contained within the lumen of the catheter. A plurality of wires connects the RF generator to the electrode. The plurality of wires extend along the length of the catheter.

### RELATED CASES

This application claims priority to U.S. Provisional Application No. 62/713,097, filed on Aug. 1, 2018 and titled “METHODS FOR HEATING DIELECTRICAL MATERIALS WITHIN A CATHETER SYSTEM,” and United States Provisional Application No. 62/713,099, filed on Aug. 1, 2018 and titled “METHODS FOR SEVERABLE COIL ASSEMBLY WITH ADJUSTABLE DETACHMENT ZONE,” which are hereby incorporated by reference herein in their entirety.

### FIELD

### Technical Field

The present disclosure relates generally to the field of delivery systems for a patient vascular system. More particularly, some embodiments relate to catheter systems that are able to produce a high radio frequency electromagnetic field.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 is a schematic view of a patient's vasculature.

FIG. 2 is a schematic view of a catheter system that produces an electromagnetic field.

FIG. 3 is a schematic view of the catheter system of FIG. 2 that cuts a dielectric material.

FIG. 4A is a schematic view of a catheter system with an embolic coil.

FIG. 4B is a schematic view of the catheter system of FIG. 4A cutting the embolic coil with the electromagnetic field.

FIG. 5A is a schematic view of a catheter system with an embolic coil with biocompatible dielectric joints.

FIG. 5B is a schematic view of the catheter system of FIG. 5A cutting the embolic coil at one of the biocompatible dielectric joints.

### DETAILED DESCRIPTION

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical and thermal interaction. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive).

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

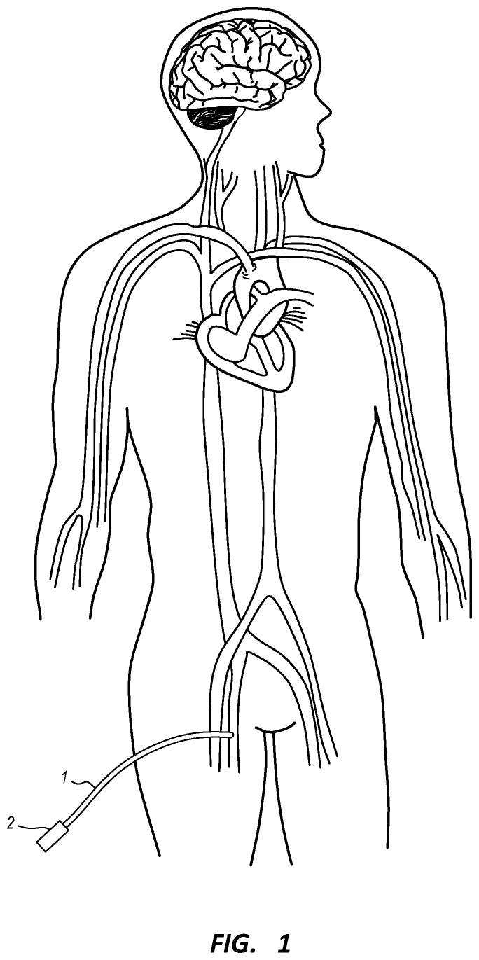

The present disclosure is directed to delivery systems that produce a high radio frequency (“RF”) electromagnetic field for various treatment procedures. FIG. 1 illustrates that a catheter delivery system may is directed to a predetermined location within a patient's vasculature. In other words, a targeted anatomy location of the patient. Such locations may include aneurysms, vasculature, and the like. The delivery system can be used in the arterial and venous peripheral vasculature, cardio vasculature, and neurovasculature.

The methods for heating dielectrical materials within a catheter system is an alternative method for the delivery and heat transfer of dielectrical materials into a vascular space. The catheter system is supplied energy via an RF (Radio Frequency) generator which delivers RF signals, through the construction of the catheter, to electrodes that are an integral component of the catheter. For example, the electrodes may be disposed within a wall of the catheter. The electrodes can be opposing plates or a ring or a split ring. The signal produced within the confines of the electrodes create an EM electromagnetic field which transfers energy, in the form of heat, to any dielectric material that passes through the EM field of electrodes. This system utilizes a catheter platform with similar construction materials and dimensions as commonly available cardiovascular, neurovascular, and peripheral vascular catheters.

Current methods of heating dielectric materials with a catheter-type of device are used, for example, in the ablation of tissue near the tip of the outer surface of said catheter where an EM field is generated. No systems currently have the ability to generate an EM field within the lumen of the catheter for heating a dielectric material. This invention has more than one application and potentially several unidentified applications for use. One application for use of this system would be to retract tissue within the lumen of the catheter, for example, to ablate the tissue. This would serve to minimize risk involved with accidental surrounding tissue damage that can occur from the currently available ablation catheters on the market, which in-turn, would minimize litigation costs, patient harm, and promote safety. Another application for use of this system would be to separate a retractable vascular implant form its delivery system by heating a heat-sensitive dielectric member to cause the member to break into two pieces. This would serve as a steady-state method for deploying vascular implants that have been delivered to very sensitive areas and areas that require a high accuracy of deployment where patient safety is of utmost concern.

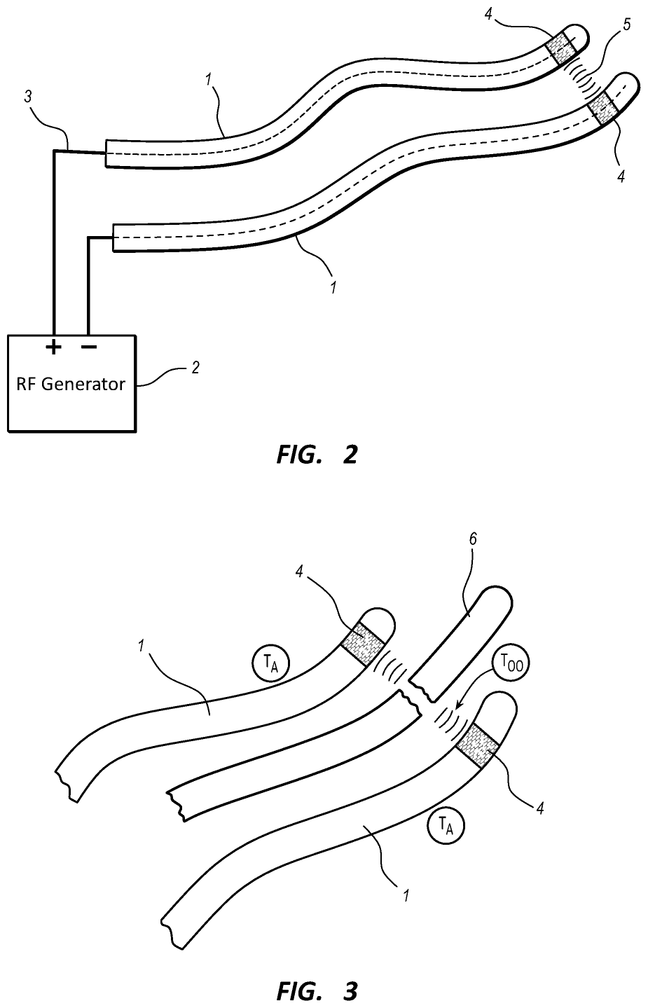

FIGS. 2 and 3 illustrate an exemplary catheter system. The methods for heating dielectrical materials within a catheter system are comprised of a catheter (1), and an RF generator (2). The dielectric materials may include, for example: Polymers/Co-Polymers/Flouropolymers such as PTFE (Teflon), PE (Polyethylene, Polyimide, Polypropylene, Polystyrene; Metal Alloys/Metal Alloy Oxides such as Titanium dioxide, Strontium titanate, Barium titanate, Titanium, NiTi (Nickel-Titanium) Tantalum, Gold, Silver, Tin, Platinum, Ptlr (Platinum-Tungsten), Ptlr (Platinum Iridium); and/or other dielectrics such as Silica. In addition, or in other embodiments, a blend of polymers, silica, and the Alloys listed may be blended to create a dielectric material suitable for use with the delivery system.

The catheter may comprise a central lumen that extends from a proximal end to a distal end of the catheter and has an opening at the distal end of the catheter. The catheter system will have electrode wires (3) terminated to electrodes which can be opposing plates, or a ring or a spilt ring (4) that is positioned distally in the catheter shaft (5). The ring may encircle the distal end of the lumen of the catheter. The spilt ring may partially encircle the distal end of the lumen of the catheter, with a first portion of the spilt ring disposed on an opposing radial side of a second portion of the spilt ring. The opposing plates may be disposed on opposing radial sides of the distal end of the catheter. When the catheter system is connected to the RF generator capable of delivering frequencies between 5 MHz-100 MHz, an electromagnetic field (5) is induced within the confines of the electrode ring which transfers a theoretical infinite temperature heat load (T_{∞}) to a suitable dielectric material (6) capable of causing ablation, melting, or vaporizing all without affecting the ambient temperature within the catheter or outside of the catheter (T_{A}). The electromagnetic field is confined within the lumen of the catheter. FIG. 3 illustrates a dielectric material that is cut or broken by the electromagnetic field. In some embodiments, a vascular implant may be separated from the dielectric material to enable a user to implant the device in a patient's vasculature.

The catheter system may also be used for to implant an embolic device for the occlusion of endovascular malformations, aneurysms, and arteriovenous malformations. The embolic device in this invention may a coil or an assembly of coils that can be sequentially detached along pre-specified points along the coil length or at infinite points along the coil length or at infinite points along the coil length. Each method as described, relies upon a high RF electromagnetic field to induce heating in a heat sensitive member. The first method for detachment utilizes a novel RF reactive coil of a fixed length that can be detached at any point along its path by use of the RF signal. The coil may be fabricated from a polymer, co-polymer, or flouro-polymer or a polymer-alloy filament or polymer-silica. When the coil is exposed to the RF signal, an energy transfer in the form of heat is transferred to the heat sensitive coil causing the coil to separate at the desired location (where the coil intercepts the RF coil). The next method for detachment utilizes an assembly of bare platinum coils which are sequentially attached, fore and aft, with a novel RF reactive carbon-polymer filament. When the filament zone between each coil is exposed to the RF signal, an energy transfer in the form of heat is transferred to the heat sensitive filament causing the coil to separate at the desired location.

Current methods to detach embolic coils only allow for the deployment of a single embolic coil of a known length. No systems currently have the ability to detach a single length of coil at any sequential point along a coil length. Additionally, current methods to detach embolic coils and other implantable vascular devices employ an electric current to cause an electrolytic response to corrode an electrode to detach the coil or related device. See U.S. Patent Application Publication No. 2005/0021023. Another method of detachment of embolic coils and related devices is through a mechanically detachable interface between the device and the delivery system. See U.S. Pat. No. 8,328,860. Another method utilizes an electrolytic severable coil assembly with a movable detachment point. See U.S. Pat. No. 5,522,836.

The mechanically detachable system presented lends to reliable detachment methods for detaching a single embolic device, which requires multiple devices to completely embolize and occlude a cerebral aneurysm for example. Furthermore, the method that utilizes an electrolytic response with a movable detachment point would prove to be an unreliable method of detachment as there is very little control over the pathway of the current through the coil device. Patient grounding may also be an issue. In some cases where blood flow is restricted to the targeted anatomy there would be an open circuit situation and electrolysis would not be possible. A system that can detach an embolic coil of infinite length at any desired point along the length of the coil could inherently de-escalate risks in regards to patient safety, decrease the time spent in a vascular interventional procedure, reduce costs for medical institutions in the quantity of devices and the stocking of those devices, and reduces costs for medical institutions and insurance agencies in both the time that a patient is in surgery and recovery.

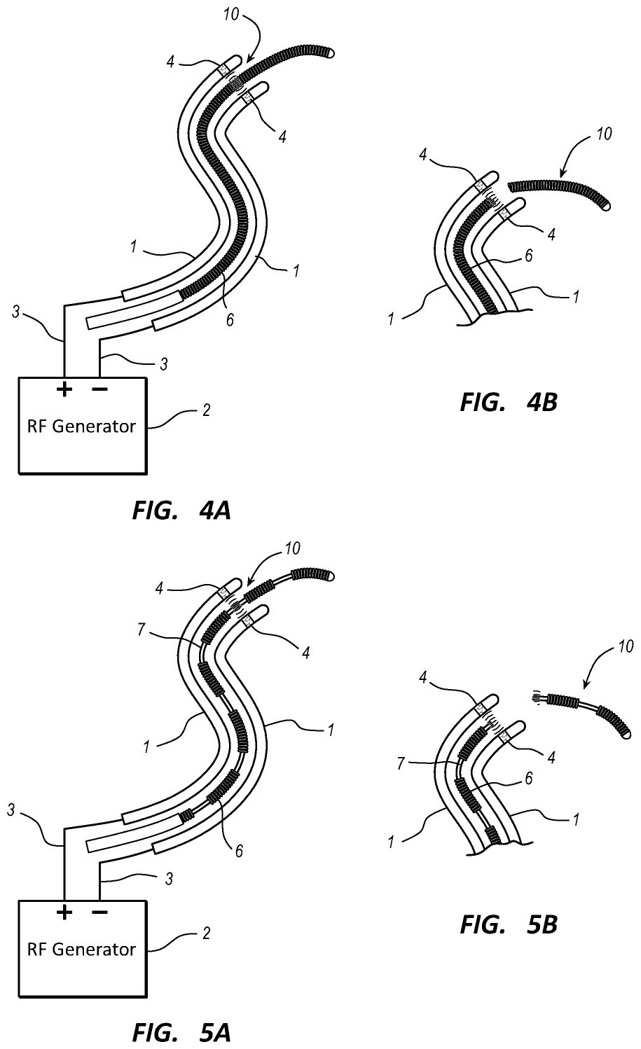

FIGS. 4A and 5B illustrate catheter systems for placing embolic coils. The catheter system includes a catheter (1) and an RF generator (2). The catheter system will have electrode wires (3) terminated to electrodes which can be opposing plates, or a ring or a spilt ring (4) that is positioned distally in the catheter shaft (5). FIGS. 4A and 4B illustrate a catheter system in which the embolic coil is fabricated from a biocompatible dielectric material (6), such as one or more of the dielectric materials discussed above. FIG. 4B illustrates the electromagnetic field cutting the embolic coil. FIG. 5A and 5B illustrate a catheter system in which the embolic coil that is fabricated from a biocompatible alloy that has a plurality biocompatible dielectric joints (7) arranged in series along the length of the embolic coil. FIG. 5B illustrates the electromagnetic field cutting the embolic coil at one of the biocompatible dielectric joints.

The delivery system with pre-attached embolic coil is employed through the catheter to the targeted anatomy which has a distal electrode ring (4) capable of emitting a high frequency electromagnetic field (10) when attached to the external RF generator capable of delivering frequencies between 5 MHz-100 MHz through the catheter electrode ring.

The application of the method for dielectric detachment may be applied to other applications including stent occlusion devices, occlusion filters, drug eluding stents, and any other vascular deliverable devices that must be retained in the vascular system for any length of time.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

1. A catheter system comprising:
a catheter with a lumen and extends from a proximal end to a distal end of the catheter, the distal end of the catheter comprising an opening;
an RF generator configured to produce an high radio frequencies;
an electrode disposed within a wall of the catheter at the distal end of the catheter, wherein the electrode is configured to produce a high radio frequency electromagnetic field contained within the lumen of the catheter; and
a plurality of wires that connect the RF generator to the electrode, wherein the plurality of wires extend along the length of the catheter.

2. The catheter system of claim 1, wherein the electrode comprises a ring that encircles the distal end of the catheter.

3. The catheter system of claim 1, wherein the electrode comprises a spilt ring that partially encircles the distal end of the catheter, wherein a first portion of the spilt ring is disposed an opposing radial side of a second portion of the spilt ring.

4. The catheter system of claim 1, wherein the electrode comprises a pair of electrodes that disposed on opposing radial sides of the distal end of the catheter.

5. The catheter system of claim 1, wherein the RF generator delivers frequencies that range between 5 MHz and 100 MHz.

6. The catheter system of claim 1, further comprising a dielectric material that is disposed within the lumen of the catheter.

7. The catheter system of claim 1, further comprising an embolic coil that is configured to extend along the length of the catheter and out of the opening of the catheter.

8. The catheter system of claim 7, wherein the embolic coil is fabricated from a biocompatible dielectric material.

9. The catheter system of claim 7, wherein the embolic coil is fabricated from one or more materials selected from a group comprising a polymer, a co-polymer, a flouro-polymer, a polymer-alloy filament, and a polymer-silica.

10. The catheter system of claim 7, wherein the embolic coil comprises a series of biocompatible dielectric joints that are spaced along a length of the embolic coil.

11. A method of dielectric detachment of an embolic coil comprising:
advancing a catheter of a catheter system to a predetermined location, the catheter system comprising:
the catheter with a lumen and extends from a proximal end to a distal end of the catheter, the distal end of the catheter comprising an opening;
an RF generator configured to produce an high radio frequencies;
an electrode disposed within a wall of the catheter at the distal end of the catheter, wherein the electrode is configured to produce a high radio frequency electromagnetic field contained within the lumen of the catheter; and
a plurality of wires that connect the RF generator to the electrode, wherein the plurality of wires extend along the length of the catheter;
advancing an embolic coil through the lumen of the catheter and out the opening;
placing the embolic coil within an aneurysm to occlude the aneurysm; and
creating an electromagnetic field to cut the embolic coil.

12. The method of claim 11, wherein the RF generator delivers frequencies that range between 5 MHz and 100 MHz.

13. The method of claim 11, wherein the embolic coil is fabricated from a biocompatible dielectric material.

14. The method of claim 11, wherein the embolic coil comprises a series of biocompatible dielectric joints that are spaced along a length of the embolic coil, wherein the embolic coil is cut at one of the dielectric joints by the electromagnetic field.

15. The method of claim 11, wherein a user determines the location to cut the embolic coil.

16. A method of producing an electromagnetic field within a lumen of a catheter comprising:
advancing a catheter of a catheter system to a predetermined location, the catheter system comprising:
the catheter with a lumen and extends from a proximal end to a distal end of the catheter, the distal end of the catheter comprising an opening;
an RF generator configured to produce an high radio frequencies;
an electrode disposed within a wall of the catheter at the distal end of the catheter, wherein the electrode is configured to produce a high radio frequency electromagnetic field contained within the lumen of the catheter; and
a plurality of wires that connect the RF generator to the electrode, wherein the plurality of wires extend along a length of the catheter;
producing a high radio frequency electromagnetic field that is contained within the lumen of the catheter.

17. The method of claim 16, further comprising retracting tissue within the lumen of the catheter and ablating the tissue with the electromagnetic field.

18. The method of claim 16, further comprising advancing a dielectric material through the lumen to the distal end of the catheter.

19. The method of claim 18, heating the dielectric material within the lumen of the catheter with the electromagnetic field.

20. The method of claim 16, wherein the electromagnetic field is configured to cause ablation, melting, or vaporizing without affect an ambient temperature within the catheter or outside the catheter.
